(19) United States
(12) Patent Application Publication
(10) Pub. No.: US 2011/0163178 A1
(43) Pub. Date: Jul. 7, 2011

(54) ODOR CONTROL UNIT WITH ESSENTIAL OILS TRAY

(75) Inventor: Timothy W. PLANKER, Cape Coral, FL (US)

(73) Assignee: HINSILBLON LABORATORIES, Fort Myers, FL (US)

(21) Appl. No.: 12/683,837

(22) Filed: Jan. 7, 2010

(51) Int. Cl. A61L 9/04; F28D 15/00; A61L 9/00

(52) U.S. Cl. 239/8; 165/104.34; 165/100; 422/124

(57) ABSTRACT

A large scale vapor dispersion apparatus for dispersing a vapor of essential oil without the use of water by forcing an air stream through and/or over a volume of the essential oil, vaporizing a portion of the essential oil, controlling the vaporization rate by increasing or decreasing the amount of heated air passing through the essential oil, and distributing the air and vapor mixture over a large scale space. The apparatus includes a blower, an evaporator assembly, piping joining the blower to the evaporator assembly, and a tray containing an essential oil. Other aspects in accordance with the present invention include a damper directing a portion of the air above and/or below the tray, and a vapor dispersing device for dispersing the air and vapor exhausted from the apparatus into a large space.

### BACKGROUND OF THE INVENTION

1. Field of the Invention

The present invention relates to an apparatus for deodorizing a stream of gas (also interchangeably referred to herein as “air”) and for large-scale dispersion of vaporized essential oils into large open spaces or volumes without the use of water.

2. Background of the Technology

Dispersion of vaporized essential oils into a large space without the use of water is known in the art, for example, by U.S. Pat. No. 6,142,383 issued to Timothy W. Planker and U.S. patent application Ser. No. 11/301,250 filed Dec. 12, 2005, by Timothy W. Planker, the contents of both of which are hereby incorporated by reference in their entirety. Additionally, U.S. Pat. No. 6,423,274, also issued to Timothy W. Planker, which is hereby incorporated by reference in its entirety, relates to an apparatus for deodorizing gas by passing the gas over a surface of a deodorizing liquid.

Although devices are known for dispersing essential oils, the devices vaporize oil by forcing a volume of air over a surface of the oil in order to vaporize or otherwise entrain the oil in the gas for subsequent dispersal or by atomizing the oil into a stream of air. While effective in some applications, there exists a need to deodorize and/or disperse essential oils at varying rates or in varying concentrations with a single device. There is also a need to more easily clean the apparatus and more easily change the mixture or type of essential oils.

### SUMMARY OF THE INVENTION

The present invention solves these problems, as well as other, by forcing warm air over and/or below a removable tray containing a volume of essential oil in order to vaporize or entrain a desired concentration of the essential oil in the air. The present invention provides for the vaporization and widespread dispersion of essential oils without necessarily using water. Further, the present invention provides the ability to easily remove and/or replace the essential oil.

According to a first aspect of the present invention, the apparatus of the present invention disperses essential oil by vaporizing a super concentrated liquid essential oil in one of three modes.

The first mode, the maximum strength setting, involves convective vaporization by forcing heated air over a surface of the liquid essential oil contained in a tray, which is located inside an evaporation chamber. The air vaporizes a portion of the liquid essential oil, wherein the resulting mixture of air and essential oil vapor is exhausted from the evaporation chamber through an outlet and possibly dispersed by a vapor dispersing device, such as a perforated hose or nozzle. The second mode, the minimum strength setting, involves conductive vaporization by forcing air below a tray containing the liquid essential oil and thereby heating the tray to heat the oil. The third mode, the medium strength setting, involves convective and conductive vaporization by forcing air both above and below the tray containing the liquid essential oil. All three modes may be initiated by adjusting a damper at the heated air inlet portion, for example, or adjusting a position of the tray inside the evaporation chamber. The resulting mixture of air and essential oil vapor is then exhausted through the outlet of the evaporation chamber and possibly dispersed by the vapor dispersing device. The present invention increases vaporization efficiency by directing the entire volume of air within the apparatus through the evaporation chamber containing the essential oil regardless of the position of the damper, or the position of the tray, as opposed to bypassing a portion of the air outside the evaporation chamber.

According to a second aspect of the present invention, the apparatus of the present invention deodorizes a flow of air used for dispersing the essential oils. The airflow passes above and/or below an essential oil, causing a portion of the essential oil to be vaporized in the air. The deodorized airflow then exits the apparatus through an exhaust pipe and possible muffler. The apparatus is useful in deodorizing air emanating, for example, from inside a pumping station of a wastewater treatment plant or air flow generated during the transport of sewage, such as when sewage is removed from a septic tank.

Additional advantages of the present invention include a low capital cost, ease of maintenance, and technological simplicity so as not to require a sophisticated electronic controller. Further, because no water is used in dispersing the essential oil and the freezing point of essential oil is well below −100° F., the apparatus of the present invention allows winter operation.

Additional aspects, advantages, and novel features of the invention will be better understood as set forth in the following description and accompanying drawings and will also become apparent to those skilled in the art upon examination of the following or upon learning by practice of the invention.

### BRIEF DESCRIPTION OF THE FIGURES

The features of the invention will be more readily understood with reference to the following description and the attached drawings, wherein:

FIG. 1 shows a side cutaway view of an odor control apparatus, in accordance with aspects of the present invention;

FIG. 2 shows a front view of an evaporator tray mounted in an evaporator cylinder of an odor control apparatus, in accordance with aspects of the present invention;

FIG. 3 shows a perspective view of an evaporator cylinder of an odor control apparatus, in accordance with aspects of the present invention;

FIG. 4 shows a side view of the odor control unit of FIG. 1 with the damper set to direct air flow below the tray, in accordance with aspects of the present invention;

FIG. 5 shows a side view of the odor control unit of FIG. 1 with the damper set to direct air flow above the tray, in accordance with aspects of the present invention;

FIG. 6 shows a side view of the odor control unit of FIG. 1 with the damper set to direct air flow over and under the tray, in accordance with aspects of the present invention;

FIG. 7 shows an overhead view of the odor control unit of FIG. 1, in accordance with aspects of the present invention; and

FIGS. 8A-8C show side views of an odor control unit, in accordance with aspects of the present invention.

### DETAILED DESCRIPTION

The detailed description may include specific details for illustrating various aspects of an odor control unit and related systems and methods. However, it will be apparent to those skilled in the art that aspects of the invention may be practiced without these specific details. In some instances, previously described or well known related elements may be shown in block diagram form, or omitted, to avoid obscuring the inventive concepts presented throughout this disclosure.

Various aspects of an odor control system, for example, may be illustrated by describing components that are coupled together. As used herein, the term “coupled” is used to indicate either a direct connection between two components or, where appropriate, an indirect connection to one another through intervening or intermediate components. In contrast, when a component is referred to as being “directly coupled” to another component, there are no intervening elements present.

Relative terms such as “lower” or “bottom” and “upper” or “top” may be used herein to describe one element's relationship to another element from the perspective illustrated in the drawings. It will be understood that relative terms are intended to encompass different orientations of an apparatus in addition to the orientation depicted in the drawings. By way of example, if an apparatus in the drawings is turned over, elements described as being on the “bottom” side of the other elements would then be oriented on the “top” side of the other elements. The term “bottom” can therefore encompass both an orientation of “bottom” and “top” depending on the particular orientation of the apparatus.

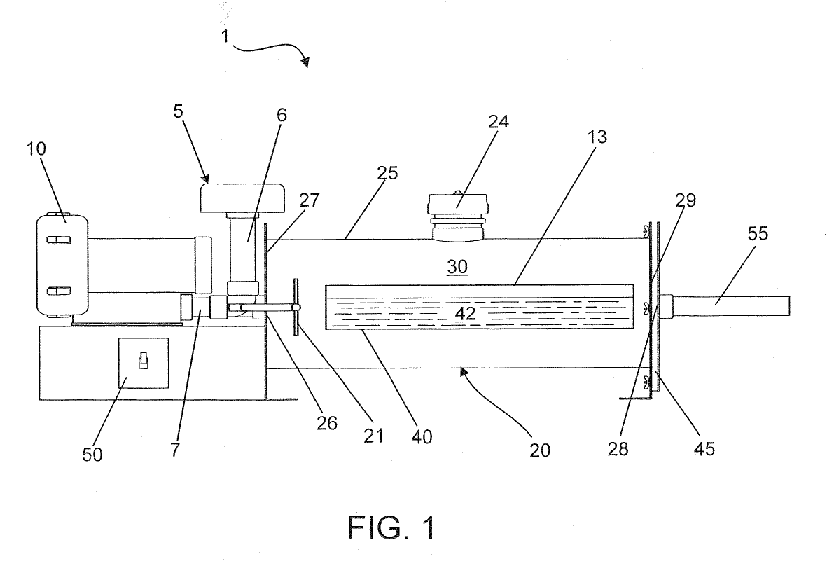

FIG. 1 provides a cutaway view of an odor control apparatus 1 for the vaporization and widespread dispersion of essential oils without the use of water, in accordance with aspects of the present invention. As shown in FIG. 1, the apparatus 1 may include an air filter assembly 5, an air blower 10, and an evaporator assembly 20.

The air filter assembly 5 may contain, for example, a replaceable, pleated paper filter cartridge that is enclosed at a top of the air filter 5 by a standard air filter cover. An air inlet pipe 6 couples the air filter assembly 5 to an inlet side of the air blower 10. The air blower 10 may be a regenerative air blower, for example, in which the air entering the air blower 10 from the air inlet pipe 6 is warmed by the heat of compression. The warm air exits an outlet side of the air blower 10 through an outlet pipe 7 that couples the air blower 10 to the evaporator assembly 20. A control panel 25, such as an electronic controller or an on/off switch, for example, may be provided to control the air blower 10. Although FIG. 1 illustrates the control panel 50 as being separate from the air blower 10, it is within the scope of the present invention that the control panel 50 could be directly attached to the air blower 10.

The evaporator assembly 20 may include an evaporator cylinder 25 having an air inlet portal 26 provided on an end wall 27 and an air outlet portal 28 provided on a second end wall 29. The evaporator cylinder 25 entirely encloses a volume of air, creating an evaporation chamber 30 inside the evaporator cylinder 25. Although described herein as having a cylindrical shape, the evaporator cylinder 25 may be any suitable shape, such as a rectangular box, a cylinder, a cube, or any other shape. The evaporator cylinder 25 can be made of any stable non-reactive or low reactive material such as, for example, stainless steel, tin, aluminum, titanium, plastic, glass, etc.

An evaporator tray 40 is located within the evaporation chamber 30. The evaporator tray 40 may be removeably positioned in the evaporation chamber 30 such that air traveling through the evaporation chamber 30 can pass both above and below the tray 40. The tray 40 has an open top and fits side to side within the evaporator cylinder 25. The tray 40 is configured to receive and hold a volume of essential oil (referred to hereinafter as “oil 42”), such as a biodegradable essential plant oil blend, for example. As shown in FIG. 1, an optional filling inlet 24, may be provided on the evaporator cylinder 25 to provide easy access for refilling the oil 42 without having to remove the tray 40 from the evaporation chamber 30. The tray 40 may be made of any suitable material that is non-reactive or low reactive. For example, the tray 40 may be made from stainless steel, tin, aluminum, titanium, plastic, etc. The material should be very heat conductive. The tray 40 may be removed from the evaporation chamber 30 by means of an access door 45, for example. The access door 45 may be attached to the evaporator cylinder 25 by any suitable fastening fixtures, including bolts and wingnuts, hinges, and/or clasp locks, for example. Other features in accordance with aspects of the present invention may include seals or sealing material provided in connection with the access door 45, for example, to enable or enhance an airtight environment in the evaporation chamber 30, providing increased internal pressure and temperature control.

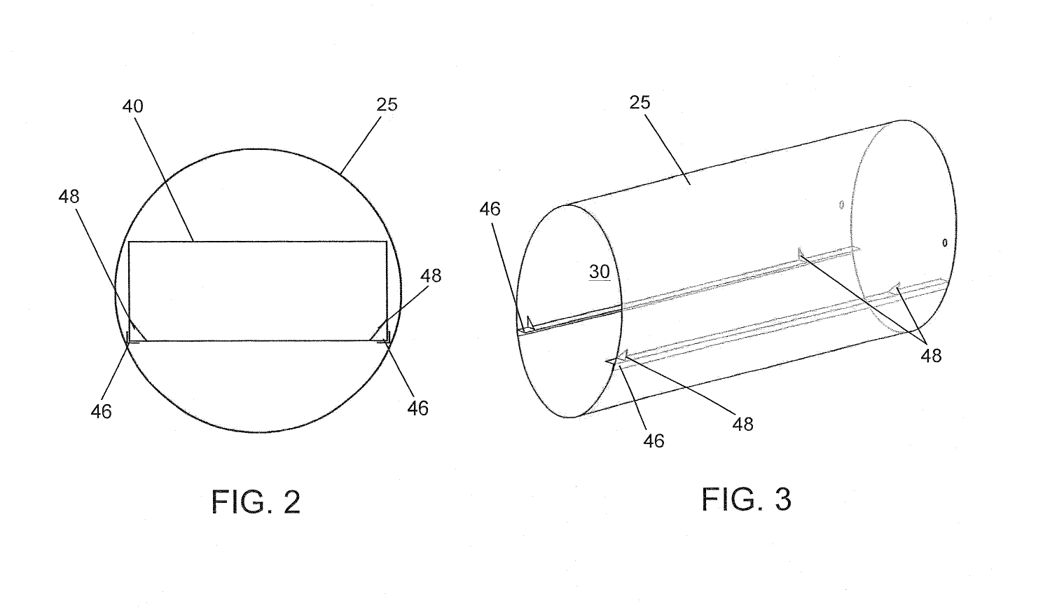

As shown in FIGS. 2 and 3, tray tracks 46, such as angle irons, for example, may be welded or otherwise attached to the evaporator cylinder 25 to cradle, or otherwise support, the evaporator tray 40 in the evaporation chamber 30. Furthermore, another feature in accordance with aspects of the present invention may be inclusion of tray stoppers 48 provided, for example, on the inlet side and/or outlet side of the evaporator cylinder 25 to work in combination with the tray tracks 46 to ensure proper positioning of the tray 40 within the evaporation chamber 30. Thus, the tray 40 can be easily replaced, drained, filled and/or cleaned.

Referring again to FIG. 1, warm air is received into the evaporation chamber 30 from the air blower 10 through the inlet portal 26 and flows over and/or under the evaporator tray 40. Odor neutralizing waterless vapor flows from the evaporation chamber 30 by way of the outlet portal 28 and into an exhaust pipe 55 coupled to the outlet portal 28. A vapor dispersing device for dispersing vaporized oil in a fog-like manner may be coupled to the exhaust pipe 55. Alternatively, the vapor dispersing device may attach directly to the outlet portal 28, for example. The vapor dispersing device can be for example a nozzle, such as those manufactured by Bete Fog Nozzle Co. The nozzle includes, for example, a ¼ inch nozzle orifice and is made of polypropylene, for example. However, it is within the scope of the present invention to include any device capable of dispersing a vaporized liquid.

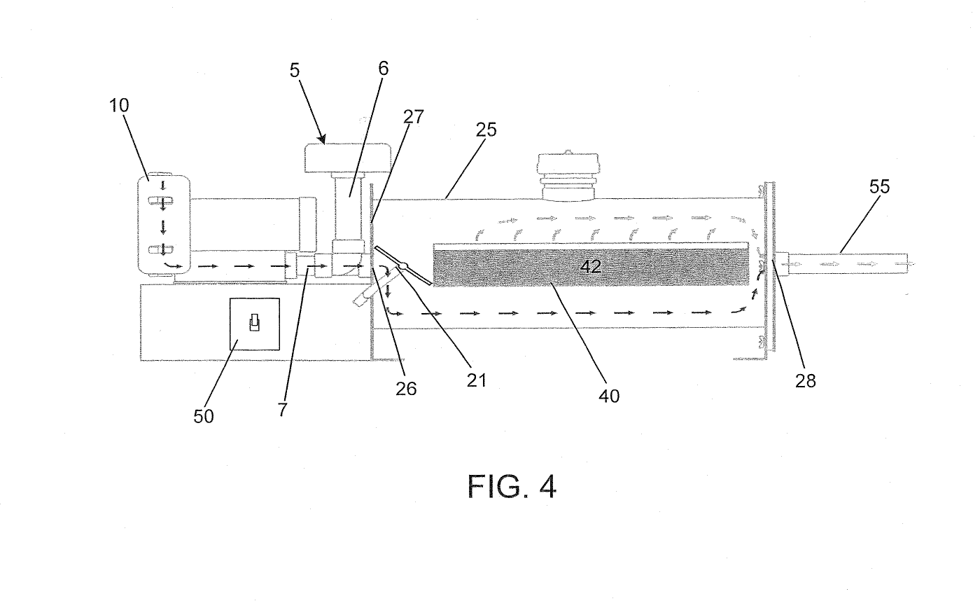

As shown in FIG. 1, a pivoting damper 21 extends from the end wall 27 of the evaporator cylinder 25. FIG. 4 shows the damper 21 in a downwardly extending position from a point on the end wall 27 above the inlet portal 26 to a bottom edge of the tray 40. When the damper 21 is arranged in such a manner, the flow of air entering the evaporator cylinder 25 is blocked from traveling above the tray 40. Rather, the air flow is routed below the tray 40. Forcing the air flow below the tray 40 allows for a conductive heating of the tray and thus, a certain minimum amount of oil 42 to evaporate into the evaporation chamber 30. Thus a mixture of air and oil 42 is produced (interchangeably referred to hereinafter as “mixture”). The combined stream of oil and air is free to flow through the outlet portal 28.

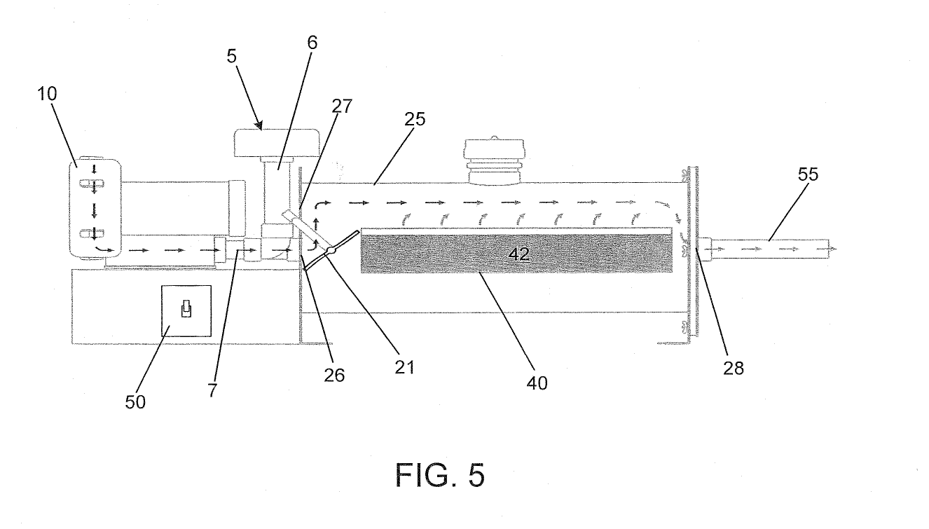

Regarding FIG. 5, a second position is shown wherein the damper 21 is positioned so that the air flow is forced to flow above the tray 40. In this position, the damper 21 extends from the end wall 27 at a point below the inlet portal 26 to the upper edge of the tray 40. By being positioned in such a manner, the path below the tray 40 is blocked and the air is forced to flow above the tray 40. When passing over the top of the tray 40, the oil 42 is in direct contact with the air and thus a certain maximum amount of oil 42 is evaporated into the air stream. The combined stream of oil and air is free to flow through the outlet portal 28. The minimum and maximum amounts of oil evaporated depend upon the temperature of the air flowing through, the temperature of the oil, and the materials of construction, the volume of air flow, and the particular mixture of oils.

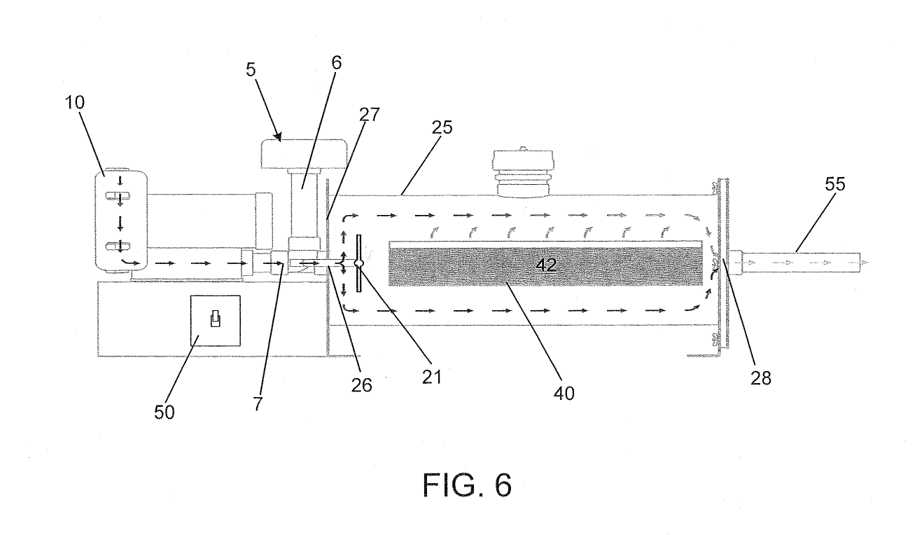

Regarding FIG. 6, a third position is shown wherein the damper 21 is positioned so that the air is free to flow both above and below the tray 40. As shown in FIG. 6, the damper 21 is positioned such that it extends parallel to the end wall 27. When in this position, neither path is totally blocked by the damper 21 and the air is free to flow both above and below the tray 40. By passing both above and below the tray 40, the oil 42 is evaporated both directly and, indirectly. Thus, a medium concentration of oil 42 is evaporated into the air stream. The combined stream of air and vaporized oil 42 is free to flow through the outlet portal 28.

It is also within the scope of the invention that the damper 21 can be positioned at any point in between the three positions described above. In these intermediate positions the ratio of air flow above the tray to the air flow below the tray will vary between the extremes described. Thus, a range of oil vapor concentrations may be achieved by adjusting the position of the damper 21. In order to maintain the damper 21 in the desired intermediate position, any known means that prohibit movement of the damper 21 can be used. For example, a locking mechanism that prevents rotation of an actuation arm may be used.

In accordance with aspects of the present invention, the air blower 10 includes a motor (not shown), such as a TEFC (Totally Enclosed, Fan Cooled) motor, having a stock size, such as a ½ horsepower (HP), ¾ HP, 1 HP, 3 HP, or 5 HP motor, for example. The motor may include permanently sealed ball bearings, for example. An air blower 10 having a motor with a more powerful motor is capable of dispersing a greater volume of vaporized oil 42 during a given time period, whereas an air blower 10 having a motor with a less powerful motor disperses a lower volume of oil 42 during the same period of time. Additionally, the inlet and outlet of the air blower 10 may be muffled so as to meet or exceed OSHA noise standards, for example.

In operation, the air blower 10 may be switched on via the control panel 50 and draws outside air through the air filter assembly 5 and air inlet pipe 6. The ambient air, heated by the compressive action of the air blower 10, may then be forced through the outlet pipe 7.

Heated air entering through the inlet portal 26 passes into the evaporation chamber 30 of the evaporator cylinder 25 and, depending on the position of the damper 21, will travel over, below, or simultaneously over and below the tray 40, causing a portion of the oil 42 to vaporize. Consequently, the capability to redirect a portion of the heated air over the tray 40, under the tray 40, or simultaneously over and under the tray 40, thereby allows for a desired concentration of the vaporized oil 42 to be exhausted from the apparatus.

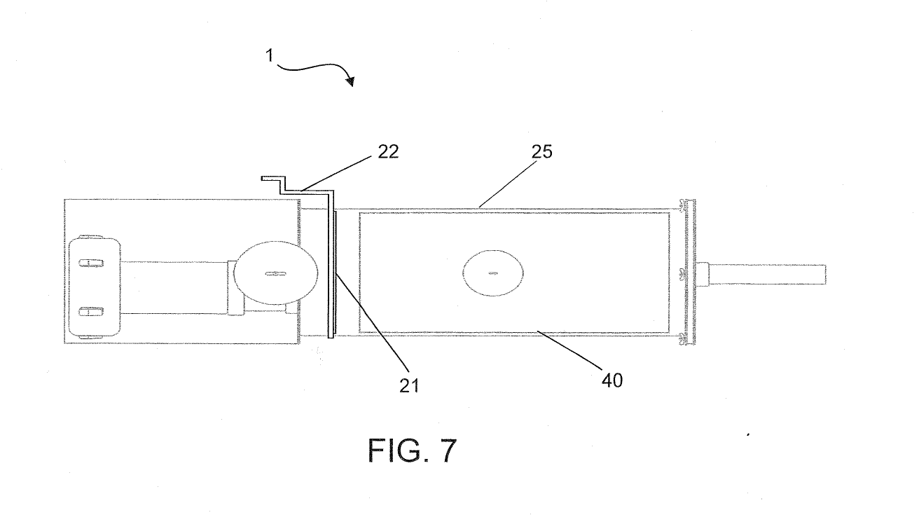

FIG. 7 shows an overhead view of the apparatus 1. By adjusting the position of the damper 21 by means of an actuating arm 22, for example, the operator can select between the three positions described above. Thus, at least three levels of oil concentration can be produced through the operation of a single apparatus. Furthermore, as discussed above, the operator can set the damper 21 at any position intermediate to the three described positions by pivoting the damper 21 in order to achieve a desired concentration of oil in air. Whenever the operator of the device wishes to change the concentration of the oil 42 in the air, the actuating arm 22 is rotated on a central axle/axis to move the damper 21 into the desired position. It is to be understood that the actuating arm is simply one example of a means for adjusting the damper position. Any other device that allows the damper 21 to rotate about a pivot axis can be used. For example, a wheel, a motor, a crank, etc., can be used.

In accordance with yet other aspects of the present invention, alternative configurations may be used to achieve the same results as having a damper 21 direct air flow from a single pipe. For example, dual pipes may be used to initially separate the air flow into two streams prior to entering the evaporator cylinder 25. The air outlet pipe 7 could first split into two pipes rather than a single central pipe, as currently shown in FIG. 1. Each of the dual pipes may have valves to control the respective flow rates of air exiting the pipes into the evaporator cylinder 25. The first pipe would open into the evaporator cylinder 25 at a point above the tray 40 and the second pipe would open into the evaporator cylinder 25 at a point below the tray 40. In this alternative, the damper 21 would not be necessary or could be set in either the maximum or minimum flow setting as described above in order to prevent the flow of air from the first pipe and the second pipe from combining before the passing over and/or below the tray 40. A single actuating device may be used to synchronously open and close the valves in order to adjust the respective flow rates from the dual pipes. When the actuating device is at one extreme, the valve in the pipe above the tray would be completely open and the valve in the pipe below the tray would be completely closed. This will cause the air to flow only above the tray because the damper has blocked the path below the tray. This setting corresponds to the maximum strength setting of the single pipe method described above. As the actuating device is adjusted, the valve in the top pipe will begin to open and the valve in the bottom pipe will begin to close. Each one of these points would decrease the concentration of oil in the air stream because less air will flow from the top pipe and more will flow from the bottom pipe. When the actuating device is in the middle setting, both valves are opened half-way, so half the gas will flow out top pipe and half will flow out the bottom pipe. This setting replicates the medium setting of the single pipe method described above. Finally, when the actuating device is set at the other extreme, the valve in the top pipe may be completely closed and the valve in the bottom pipe will be completely open. This will cause the air to only flow out the bottom pipe and below the tray. This setting corresponds to the minimum setting in the single pipe method.

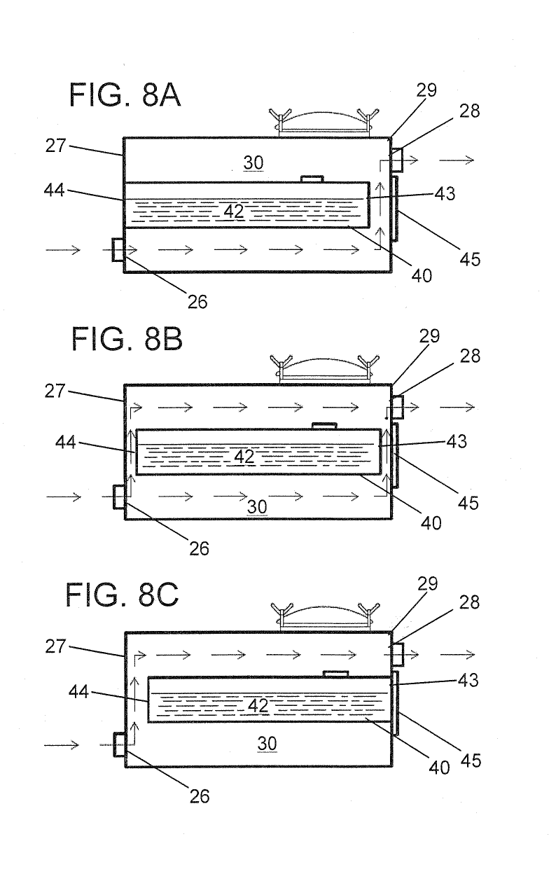

FIGS. 8A-8C illustrate aspects of yet another configuration of the apparatus in which the tray 40 may be configured to slide within the evaporator cylinder 25 to control the air flow above and below the tray 40. For example, as shown in FIG. 8A, the tray 40 may be extended longitudinally into the evaporator cylinder 25 through the access door 45 so that an end surface 44 of the tray abuts an interior surface of the end wall 27 of the evaporator cylinder 25. The tray 40 may be formed to have a lateral dimension equal to or slightly smaller than an interior lateral dimension of the evaporator cylinder 25. Thus, insertion of the tray 40 into the evaporator cylinder 25 substantially or wholly prevents air flow leakage along the longitudinal length of the sides of the tray 40 and along the end surface 44. Sealing structures, including rubber lubricant seals, for example, in combination with the tray tracks, for example, may be used to further enhance the air flow properties of the apparatus, in accordance with aspects of the present invention.

As shown in FIG. 8A, with the end surface 44 of the tray 40 abutting the interior surface of end wall 27 of the evaporator cylinder 25, warm air from the air inlet portal 26 may be directed below the tray 40 upon entering the evaporator chamber 30. Accordingly, air flow is routed substantially below the tray 40. Forcing the air flow below the tray 40 allows for a conductive heating of the tray and thus, a certain minimum amount of oil 42 to evaporate into the air stream. Thus a odor neutralizing mixture of air and vaporized oil 42 is produced (interchangeably referred to hereinafter as “mixture”). The odor neutralizing waterless mixture is free to exit the evaporation chamber 30 by flowing through the outlet portal 28.

Regarding FIG. 8B, a second position is shown wherein the tray 40 is positioned to allow a gap between the end surface 44 of the tray 40 and the interior surface of the end wall 27. Accordingly, the tray is in a position so that the air is free to flow both above and below the tray 40. When the tray 40 is placed in this position, air is permitted to flow through the gap, as well as below the tray 40, creating an air flow both above and below the tray 40. By passing both above and below the tray 40, the oil 42 is evaporated both directly and indirectly. Thus, a medium concentration of oil 42 may be evaporated into the air stream. The combined streams of air and vaporized oil 42 are then free to flow through the outlet portal 28.

FIG. 8C illustrates a third position in which the tray 40 is positioned so that a second end 43 abuts an interior surface of the second end wall 29 and/or an interior surface of the access door 45. In this manner, the warm air flow is forced to flow above the tray 40. With the tray 40 positioned in such a manner, the path below the tray 40 is blocked and the air below the tray 40 is prevented from exiting the evaporator chamber 30 through the exit portal 28. Rather, the air is forced to flow above the tray 40. Thus, when passing over the top of the tray 40, the oil 42 is in direct contact with the air and a certain maximum amount of oil 42 may be evaporated into the air stream. The combined stream of oil and air is then free to exit the evaporation chamber 30 by flowing through the outlet portal 28. The minimum and maximum amounts of oil evaporated depend upon the temperature of the air flowing through, the temperature of the oil, and the materials of construction, the volume of air flow, and the particular mixture of oils.

In accordance with other aspects of the present invention, various sensors and indicators may be provided, which may also be connected to the controller 50, for example, to provide important data for control of the system. For example, a concentration indicator may be included. The concentration indicator would display either a number or another visual indicator to show how much oil will be in the air stream upon exiting the evaporation chamber 30. A looking glass or a level float, for example, may be provided to manually or mechanically provide a means for determining the level of oil 42 in the tray 40. Pressure and temperature gauges may be provided that indicate or communicate to the controller 50, for example, the internal pressure and/or temperature of the evaporation chamber 30. Adjustments and/or maintenance, such as filter replacement, may be performed based on the readings of the various gauges. For example, a low system temperature and/or pressure reading may indicate that the system is not operating efficiently. The filter may be clogged and/or, if the unit is enclosed in a cabinet, for example, the cabinet vents may be checked for obstructions. A high temperature or pressure reading may indicate an obstruction in the outlet section of the evaporator assembly, or a problem with the vapor distribution system, for example.

Example uses of the present invention include deodorizing an air flow generated by pumping waste material from a septic tank or other waste facility to a tank, such as a tank mounted on a truck. Additionally, the present invention is useable to deodorize an air flow emitted from a pump house of a wastewater treatment plant. Accordingly, it is within the scope of the present invention that the apparatus be portable, such as transportable by truck, to a preferred location or permanently mounted at a particular location.

While there has been described what are at present considered to be preferred embodiments of the present invention, it will be understood that various modifications may be made thereto, and it is intended that the appended claims cover all such modifications as fall within the true spirit and scope of the invention. Other modifications will be apparent to those skilled in the art.

What is claimed is:

1. An apparatus for deodorizing and dispersing a gas stream comprising:
a blower having an inlet and an outlet; and
an evaporator assembly comprising:
an evaporator cylinder having an inlet portal and an outlet portal;
an evaporator tray positioned inside the evaporator cylinder; and
a pivoting damper located between the inlet portal of the evaporator cylinder and the evaporator tray;
wherein the outlet of the blower is in communication with the inlet portal of the evaporator cylinder;
and wherein the pivoting damper directs a flow of gas entering the evaporator cylinder by blocking a flow path.

2. The apparatus according to claim 1, wherein the blower is a regenerative air blower.

3. The apparatus according to claim 1, wherein the pivoting damper is positionable between a first damper position, a second damper position and a third damper position.

4. The apparatus according to claim 3, wherein the first damper position directs the flow of gas on a path above the tray.

5. The apparatus according to claim 4, wherein the second damper position directs the flow of gas on a path below the tray.

6. The apparatus according to claim 5, wherein the third damper position splits the flow of gas into a first stream and a second stream such that the first stream travels on a path above the tray and the second stream travels on a path below the tray.

7. The apparatus according to claim 1, further comprising an access door for providing access to the evaporator tray in the.

8. The apparatus according to claim 1, wherein the tray further comprises an open top.

9. The apparatus according to claim 1, wherein the reservoir further comprises angle irons to receive the tray.

10. The apparatus according to claim 1, wherein the pivoting damper further comprises an actuating arm extending outside of the reservoir.

11. A method of waterless dispersion of essential oil comprising:
blowing a gas stream into an evaporation chamber containing a tray having a volume of essential oil;
directing the gas stream along a path within the evaporation chamber and around the tray;
entraining a portion of the volume of essential oil to form an entrained gas; and
dispersing the entrained gas into an open space.

12. The method according to claim 11, further comprising directing the gas stream along a path above the tray.

13. The method according to claim 11, further comprising directing the gas stream along a path below the tray.

14. The method according to claim 11, further comprising directing the gas into first and second streams such that the first stream travels along a path above the tray and the second stream travels along a path below the tray.

15. The method according to claim 11, wherein the gas stream is simultaneously heated and blown by a regenerative air blower.

16. The method according to claim 11, further comprising removing the tray through an access door to the evaporation chamber.

17. The method according to claim 16, further comprising returning the tray to the evaporation chamber.

18. The method according to claim 14, further comprising combining the first gas stream and the second gas stream before dispersing the entrained gas into the open space.

19. The method according to claim 12, further comprising redirecting the gas stream by repositioning a damper located between an inlet of the evaporation chamber and the tray.

20. The method according to claim 13, further comprising redirecting the gas stream by repositioning a damper located between an inlet of the evaporation chamber and the tray.

21. The method according to claim 14, further comprising redirecting the gas stream by repositioning a damper located between an inlet of the evaporation chamber and the tray.

22. The method according to claim 19, wherein the damper is repositioned by rotating an actuating arm protruding from the evaporation chamber.

23. The method according to claim 20, wherein the damper is repositioned by rotating an actuating arm protruding from the evaporation chamber.

24. The method according to claim 21, wherein the damper is repositioned by rotating an actuating arm protruding from the evaporation chamber.

25. The method according to claim 12, further comprising redirecting the gas stream by repositioning the tray to abut an end wall of the evaporation chamber.

26. The method according to claim 13, further comprising redirecting the gas stream by repositioning the tray to abut an end wall of the evaporation chamber.

27. The method according to claim 14, further comprising redirecting the gas stream by repositioning the tray to provide a gap between both ends of the tray and end walls of the evaporation chamber.

28. An apparatus for deodorizing and dispersing a gas stream comprising:
a blower having an inlet and an outlet; and
an evacuation chamber having an airflow inlet and an airflow outlet; and
a tray;
wherein the outlet of the blower is in communication with the airflow inlet of the evacuation chamber; and
wherein the tray is positioned to configure an airflow path from the airflow inlet to the airflow outlet of the evacuation chamber.

29. The apparatus of claim 28, wherein the tray is positioned to direct the airflow path substantially below the tray.

30. The apparatus of claim 28, wherein the tray is positioned to direct the airflow path substantially above the tray.

31. The apparatus of claim 28, wherein the tray is positioned to simultaneously provide an airflow path above and below the tray.
